(19) United States
(12) Patent Application Publication
(10) Pub. No.: US 2006/0161064 A1
(43) Pub. Date: Jul. 20, 2006

(54) Computer-assisted detection of systolic murmurs associated with hypertrophic cardiomyopathy

(75) Inventors: Raymond L. Watrous, Belle Mead, NJ (US); Deborah M. Grove, North Brunswick, NJ (US)

Correspondence Address: RATNERPRESTIA, P O BOX 980, VALLEY FORGE, PA, 19482-0980, US

(73) Assignee: Zargis Medical Corp.

(21) Appl. No.: 11/037,665

(22) Filed: Jan. 18, 2005

(51) Int. Cl. A61B 5/0402

(52) U.S. Cl. 600/509

(57) ABSTRACT

A method for assisting in the diagnosis of heart murmurs. The method calculates a normalized measure of mid-range energy for at least one systolic or diastolic interval in a sequence of heartbeats and displays the mid-range energy measure of the systolic and/or diastolic interval in a graphical form. The sequence of heartbeats is also processed to detect and diagnose heart murmurs by adjusting a murmur count threshold responsive to the mid-range energy. The method may be used to diagnose hypertrophic cardiomyopathy and to determine effective therapeutic drug dosage or therapeutic device setting.

### FIELD OF THE INVENTION

The present invention concerns computer assisted detection of heart sounds and, in particular, the detection of systolic murmurs.

### BACKGROUND OF THE INVENTION

Systolic obstruction may produce systolic murmurs audible on auscultation. These murmurs may be associated with hypertrophic cardiomyopathy (HCM) a heart condition that is the most common cardiovascular cause of sudden death in young athletes. HCM is characterized by a systolic murmur that diminishes when a patient squats from a standing position. This murmur increases in intensity when a patient performs a Valsalva maneuver or isometric hand grip.

HCM is a relatively common autosomal dominant genetic anomaly with heterogeneous expression that is characterized by myocardial cellular disarray in various locations of the ventricles. In its obstructive form (HOCM), comprising approximately 40 percent of the cases, there is a systolic obstruction to the aortic outflow due to the proximity of the anterior leaflet of the mitral valve and the ventricular septum, enlarged and distorted by the cellular disarray.

The nonobstructive form of HCM constitutes about 60 percent of the cases and is characterized by myocardial cellular disarray in myocardial locations that do not produce obstruction and, therefore, do not produce a murmur.

Due to the prevalence of HCM, a medical family history and physical examination including auscultation of the heart are recommended by the American Heart Association (AHA) for pre-participation screening of athletes. While auscultation by a competent examiner using suitable maneuvers would be sufficient to detect the murmur of HOCM, the variability of clinical skills and uneven compliance with AHA guidelines has created a situation where young athletes with HOCM are frequently not flagged for further study before engaging in competitive sports.

Systolic and diastolic murmurs may be indicative of other heart conditions, including conditions that may be mitigated by use of medication or therapeutic devices such as pacemakers. Auscultation may be used to determine the best dosage for the medication or the best setting for the device. The best dosage or setting corresponds to the smallest murmur. Thus, the adjustment is an iterative process where different dosages or different settings are applied to a subject and, after the subject has stabilized, the murmur is measured using auscultation. Because it may take several hours for a subject to stabilize, the auscultation may be performed by different examiners. Variations among the examiners and variations in the patient between measurements, however, may make it difficult to determine the best dosage or setting.

### SUMMARY OF THE INVENTION

The present invention is embodied in a method for assisting in the diagnosis of heart murmurs using graphically displayed data. The exemplary method calculates a normalized measure of mid-range energy for at least one of systolic or diastolic intervals in a sequence of heartbeats signals and displays the mid-range energy measure of the at least one systolic or diastolic interval in a graphical form.

The invention is also embodied in a method for assisting in the diagnosis of heart murmurs by adjusting heart murmur detection based on measuring mid-range energy. The method receives an audio signal representing heart sounds and detects heart murmurs in the audio signal during a predetermined interval to develop a count of the murmurs. The method then processes the audio signal to develop a measure of mid-range energy and adjusts a murmur count threshold responsive to the measure of mid-range energy. The method diagnoses heart murmurs responsive to the murmur count and the adjusted murmur count threshold.

One aspect of the invention is a method for determining an effective dose of a therapeutic agent for treating a heart condition. According to this method, a trial dosage of the therapeutic agent is administered and an audio signal representing heart sounds is received. The method detects heart murmurs in the audio signal during a predetermined interval to develop a murmur count. The method then processes the audio signal to develop a measure of mid-range energy in the signal and adjusts a murmur count threshold responsive to the measure of mid-range energy. The method determines the effectiveness of the trial dose of the therapeutic agent responsive to the murmur count and the adjusted murmur count threshold.

Another aspect of the invention is a method for aiding in the diagnosis of cardiac murmurs associated with hypertrophic cardiomyopathy in a patient. According to this method, the presence and magnitude of cardiac murmurs in the patient is determined while the patient is in multiple postures. The presence and magnitude of cardiac murmurs is compared for the multiple postures. The method receives an audio signal representing heart sounds and detects cardiac murmurs in the audio signal during a predetermined interval to develop a murmur count. The method also processes the audio signal to develop a measure of mid-range energy of the signal and adjusts a murmur count threshold responsive to the measure of mid-range energy. The method determines the presence of cardiac murmurs in each of the multiple postures responsive to the murmur count and the adjusted murmur count threshold.

Another aspect of the invention is a method for adjusting a parameter of a therapeutic device to treat a heart condition. The method sets the parameter to a trial setting and receives an audio signal representing heart sounds. The method then detects heart murmurs in the audio signal during a predetermined interval to develop a murmur count. The method processes the audio signal to develop a measure of mid-range energy and adjusts a murmur count threshold responsive to the measure of mid-range energy. The method determines the effectiveness of the trial parameter setting responsive to the murmur count and the adjusted murmur count threshold.

### BRIEF DESCRIPTION OF THE DRAWINGS

The invention is best understood from the following detailed description when read in connection with the accompanying drawing. It is emphasized that, according to common practice, the various features of the drawing are not to scale. On the contrary, the dimensions of the various features are arbitrarily expanded or reduced for clarity. Included in the drawing are the following figures:

FIG. 1 is a functional block diagram of a cardiac diagnostic system that includes an embodiment of the present invention.

FIG. 2 is a flow-chart diagram of a cardiac diagnostic according to the present invention.

FIG. 3 is a flow-chart diagram that is useful for describing the step of measuring mid-range energy shown in FIG. 2.

FIG. 4 is a flow-chart diagram of a system for diagnosing HCM that uses an embodiment of the present invention.

FIGS. 5A and 5B are bar graphs that are useful for describing a display produced by the cardiac diagnostic system shown in FIG. 1 when using the method of diagnosing HCM shown in FIG. 4.

FIG. 6 is a flow-chart diagram of a method for adjusting dosage of a heart medication that employs an embodiment of the present invention.

FIG. 7 is a flow-chart diagram of a method for adjusting a parameter of a therapeutic device that employs an embodiment of the present invention.

### DETAILED DESCRIPTION OF THE INVENTION

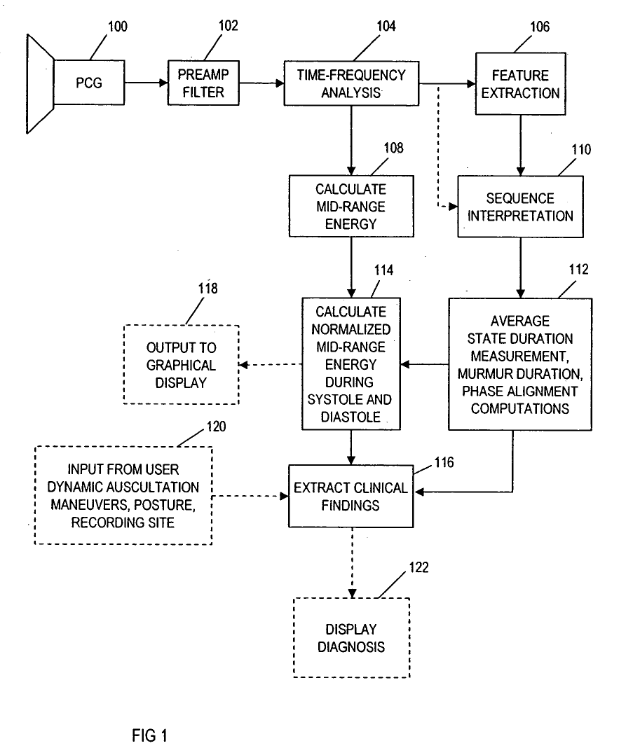

FIG. 1 is a functional block diagram of an exemplary cardiac diagnostic system according to the present invention. The system shown in FIG. 1 includes many of the elements of the system described in U.S. Pat. No. 6,572,560 entitled MULTI-MODAL CARDIAC DIAGNOSTIC DECISION AND SUPPORT SYSTEM AND METHOD, the contents of which are incorporated herein by reference for their teaching on cardiac diagnostic systems.

The present invention, however, includes additional features related to the detection and analysis of mid-range energy in acoustic heart signals. In the system shown in FIG. 1, heart sounds are detected by a phonocardiograph instrument (PCG) 100, which may be, for example, an electronic stethoscope. Output signals provided by the PCG 100 are amplified and filtered by a combination preamplifier/filter 102 to increase the amplitude of signals that are in a range of frequencies corresponding to heart sounds while attenuating signals outside of that frequency range. The preamplifier/filter 102 serves to increase the signal-to-noise ratio of the acoustic heart signals.

A time-frequency analysis circuit 104 receives the signals provided by the preamplifier/filter 102 and analyzes these signals using, for example, a wavelet decomposition to extract frequency information from the signal. Although the exemplary embodiment described below employs a wavelet transform and a Morlet wavelet, it is contemplated that other time-frequency analysis methods may be used and that other wavelets may be used. The wavelet decomposition is desirably scaled to compensate for variations in amplitude of the filtered and amplified acoustic heart sounds provided by element 102. The wavelet decomposition may be sampled logarithmically. In the exemplary embodiment, the magnitude squared wavelet coefficients are computed and scaled to compensate for logarithmic frequency spacing. The output data of the wavelet decomposition circuit is applied to a feature extraction circuit 106 and to a circuit 108 that calculates the mid-range energy in the acoustic heart sounds.

A feature extraction circuit 106 receives the signals provided by the wavelet decomposition of circuit 104 and identifies basic heart sounds, clicks and murmurs. In the exemplary embodiment, a neural network feature extraction circuit is trained from labeled examples of heart sounds. The neural network feature extraction circuit is desirably of the time-delay type, where the input layer, number of layers, unit function, and initial weight selection are appropriately chosen using well-known methods. Although a neural network of time-delay type is utilized, it is contemplated that other types of neural networks may be employed.

A sequence interpretation circuit 110 parses the extracted features from feature extraction circuit 106 using a state-transition model of the heart to determine the most probable sequence of cardiac events. The state machine may desirably be a hidden Markov model (HMM) or may be other types of state transition models. The output of the sequence interpretation circuit is applied to a duration and phase measurement circuit 112.

Time-frequency analysis circuit 104 may also extract features relevant to basic heart sounds, clicks and murmurs using MEL cepstrum signal analysis. MEL cepstrum signal analysis is well known in speech analysis. For example, see U.S. Pat. No. 6,725,190. The Mel cepstral coefficients may include total energy and first and second differences. Cepstral mean subtraction may desirably be implemented to remove channel differences such as filtering by PCG sensor 100. Features extracted by the MEL cepstrum signal analysis may alternatively be input to sequence interpretation circuit 110, shown by the dashed line.

Duration and phase measurement circuit 112 computes the average state durations of the sequence model, murmur duration and phase alignments. The output data of the duration and phase measurement circuit is applied to a normalized mid-range energy circuit 114 and to a clinical findings extraction circuit 116.

A circuit 108 that calculates mid-range energy, uses the wavelet decomposition from time-frequency analysis circuit 104 over the frequency region where the majority of heart murmurs may be found. Wavelet decomposition scales may correspond to the frequency region of 150-600 Hz or more particularly the range of 206 Hz-566 Hz. The wavelet decomposition scales of interest are summed together over the duration of the heart signal to represent the energy in the bandwidth of interest.

Mid-range energy circuit 108 represents all of the mid-range frequency energy across the entire recorded heart sound signal. The energy computed in circuit 108 may be dependent upon the recording level, signal artifacts, or heart signal transmission strength from the chest wall to PCG 100.

A normalized mid-range energy circuit 114 normalizes the mid-range energy for a desired interval. In the exemplary embodiment, systolic and diastolic intervals are of interest. The mid-range energy for each detected systolic and diastolic interval across the sequence of heartbeats is desirably normalized. A summary interval energy representing an average systolic and diastolic energy across a sequence of heart sounds is desirably computed.

Normalized mid-range energy circuit 114 data output may be transmitted to a graphical display 118. Graphical display 118 may show the mid-range energy as a function of systolic and diastolic interval and magnitude.

Duration and phase measurement circuit 112 and normalized mid-range energy circuit 114 output data are desirably applied to clinical findings extraction circuit 116. In addition, any input 120 from a user, regarding dynamic auscultation maneuvers, posture, or recording site may be applied to clinical findings extraction circuit 116.

Clinical findings extraction circuit 116 determines clinical findings based on normalized mid-range energy, state duration, phase and amplitude information. Any input 120 from a user, may be further incorporated into the extraction of clinical findings circuit 116.

In clinical findings extraction circuit 116, any cardiac murmurs present in a heartbeat are analyzed with respect to diagnosing the entire heart signal. Murmurs may be further classified relative to systolic/diastolic intervals and may be further labeled with respect to early, mid, late, pan-systolic, pan-diastolic or continuous. A graphical display 122 may be utilized to display the detection and diagnosis results.

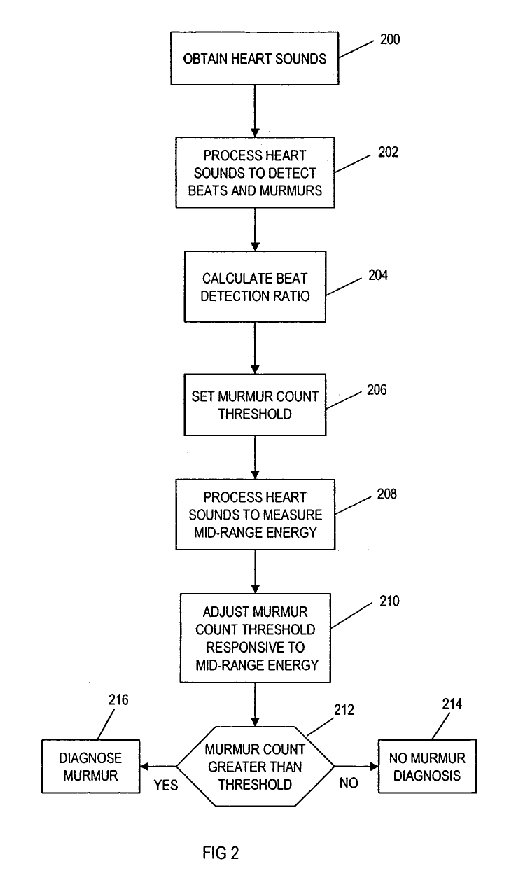

FIG. 2 shows a method for incorporating a normalized mid-range energy measure into a murmur detection algorithm for assisting in the diagnosis of heart murmurs. Heart sounds are obtained, step 200 and processed as described in FIG. 1 from PCG 100 through the duration and phase measurement circuit 112 to identify heartbeats and cardiac murmurs within the heartbeats, step 202.

Processing step 202 desirably provides a plurality of detected heartbeats. The duration of each detected heartbeat is desirably computed. A median duration for all detected heartbeats over the duration of the received heart signal may be determined. A beat detection ratio, step 204 may be computed by comparing the number of detected beats from step 202 with the number of expected heats derived from the median heartbeat duration and the heart signal duration.

Processing step 202 desirably provides a count of a number of heartbeats with murmurs detected. A murmur count may be computed by comparing the number of heartbeats with murmurs detected to the number of detected heartbeats.

A murmur count threshold, step 206, may be determined by utilizing a comparison of the beat detection ratio to the murmur count. For example, if the beat detection ratio is high, a lower murmur count may be tolerated before a murmur is diagnosed as occurring in the heart signal. If the beat detection ratio is low, a higher murmur count may be required before a diagnosis of heart murmur is allowed.

The murmur count threshold is also desirably a function of normalized mid-range energy. The heart sound signal is also processed to measure the normalized mid-range energy, step 208. An average value representing energy in the systolic and diastolic sub-intervals may be utilized for the murmur count threshold. A maximum sub-systolic or sub-diastolic energy may further be determined. Other suitable methods for utilizing mid-range energy may be utilized.

The murmur count threshold may also be a function of input 120, FIG. 1, from a user regarding dynamic auscultation maneuvers, posture or recording site. The murmur count threshold may be adjusted in response to user input. For example, if greater errors are expected to occur, based on the review of study populations, at a particular auscultation site in the standing posture, the murmur count threshold may be set increased as compared to another auscultation site and posture.

The murmur count threshold may be adjusted in response to the normalized mid-range energy, step 210. This adjustment may be in inverse proportion to the normalized mid-range energy. For example, if the beat detection ratio is lower but the mid-range energy is high, indicating the presence of a high grade murmur, the murmur count threshold may be decreased. Alternatively, the presence of a low or zero grade murmur may require a high murmur count threshold before a murmur diagnosis decision on the heart sound signal may be reached.

The normalized mid-range energy may be further converted to a murmur grade. The murmur count threshold may be adjusted in response to the murmur grade.

Final determination of overall murmur diagnosis compares the murmur count to the murmur count threshold, step 212. If the murmur count is less than the murmur count threshold, then there is no murmur diagnosis for the heart sound signal, step 214. If the murmur count is greater than the murmur count threshold, a murmur is diagnosed for the heart sound signal, step 216. While the preceding processing steps may detect murmurs in individual heartbeats, step 212 determines whether analysis results will indicate whether the heart sound signal as a whole may be diagnosed with heart murmurs.

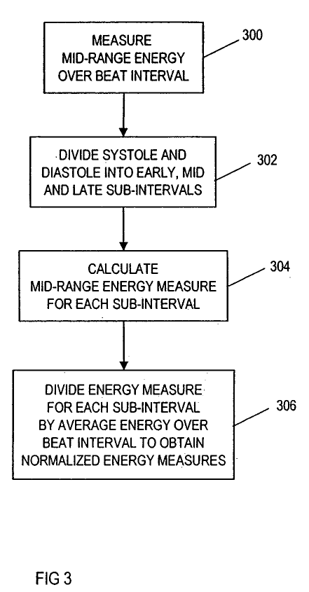

FIG. 3 represents an exemplary method of calculating the normalized mid-range energy over a plurality of sub-intervals. In the exemplary embodiment, intervals of interest are the systolic interval, defined from the end of S1 to the onset of S2, and the diastolic interval, defined from the end of S2 to the onset of S1.

In step 300, the resulting heart sound locations from duration and phase measurement circuit 112 are parsed to find systolic interval and diastolic interval timestamps from each detected heartbeat. The mid-range energy as described above is measured for all detected systolic and diastolic intervals using the parsed timestamps.

The systolic and diastolic intervals are divided into third intervals, step 302 in the exemplary embodiment. The third intervals represent the energy in the early, mid, and late portions of systole and diastole. Although the exemplary embodiment shows systolic and diastolic intervals divided into thirds, subdivision into a greater number of intervals may be of interest and is not excluded.

The sub-interval energy is calculated in step 304. Mid-range energy may be computed as described in mid-range energy circuit 108 over the sub-interval duration. Each sub-interval across the sequence of heartbeats may be represented by an average value for that sub-interval duration. The average value may be computed by the mean, median, frequency, or other methods over the duration of the interval. In the exemplary embodiment, the average value is computed from the mean.

A normalized mid-range energy measure is then computed in step 306. The mid-range energy measure for each sub-interval is divided by a normalization factor representing the nominal heart signal energy.

The normalization factor may be the nominal mid-range energy over the entire heart sound signal. The nominal mid-range energy may be computed from mean energy, median energy, frequency or by other means. In the exemplary embodiment, the median energy is calculated. In the exemplary embodiment, the nominal energy is computed from the same frequency range of interest as the mid-range energy.

The resulting normalized energy may further be presented as a logarithmic ratio or a decibel ratio. The resulting normalized energy may desirably be converted to a murmur grade based on a correlation between normalized energy to standard auscultation murmur grade. For example, a study of a population with heart murmurs, such as HCM, may be undertaken to record and analyze heart murmurs. The recordings may be further reviewed by a trained cardiologist who may assign a standard murmur grade to the study population. A mid-range energy measure may then be correlated against the cardiologist's grading of the study population to provide a translation of mid-range energy to murmur grade. The heart murmurs may be reviewed in terms of any of murmur duration, magnitude and frequency spectrum. Psychoacoustics of the heart signal may be taken into account during heart murmur review, such as the murmur appearing to be fainter in the presence of another loud sound.

After the normalized mid-range energy is computed for each subinterval it may be displayed as shown in graphical display 118 of FIG. 1. It is also desirably incorporated in a murmur diagnosis of the heart signal, by clinical findings extraction circuit 116 of FIG. 1.

Mid-range energy may be displayed graphically as a bar graph. An exemplary bar graph is shown in FIG. 5A. FIG. 5A shows the distribution of energy in the early systolic interval (ESI) 502, mid systolic interval (MSI) 504 and late systolic interval (LSI) 506. Similarly, the bar graph desirably shows the energy distribution of any early diastolic interval (EDI), mid diastolic interval (MDI) and late diastolic interval (LDI). The bar graph may show the energy level by the y axis. In the exemplary embodiment, the y-axis shows a decibel ratio, of sub-interval mid-range energy to nominal mid-range signal energy. Alternatively, this ratio may be further converted to a standard auscultation murmur grade.

Mid-range energy displayed as a bar graph desirably provides a murmur contour as well as murmur energy. In auscultation, murmur contour is important in heart disease diagnosis. Typical murmur contours may include decrescendo, crescendo-decrescendo, constant intensity and increasing intensity just prior to the onset of a heart sound. For example, in FIG. 5A, a crescendo-decrescendo type systolic murmur of some energy is indicated. A lack of energy in the early, mid and late diastolic intervals suggests the there is no diastolic murmur present in the heart signal.

Mid-range energy displayed as a bar graph also provides a means to compare murmur magnitude and contour as a function of patient posture or auscultation location. For example, a patient may be auscultated in the reclining position with a resulting mid-range energy graph of FIG. 5A showing systolic intervals 502, 504 and 506 of some sizeable energy as a crescendo-decrescendo murmur contour. The same patient may be auscultated at the same auscultation site but in the standing position, FIG. 5B. Here, murmur contour is preserved but the murmur energy has increased significantly, as shown in systolic intervals 510, 512 and 514.

Mid-range energy displayed as a bar graph desirably provides a simultaneous murmur magnitude and murmur contour. Murmur energy may be presented such that it may be correlated with, or serve as a surrogate to standard auscultatory murmur grade.

Mid-range energy is a numerical value that indicates the level of systolic and diastolic energy. Mid-range energy results are not a diagnosis of murmur pathology. A physician may use the graphical display of energy for systolic and diastolic sub-interval magnitude and contour to determine murmur pathology.

The diagnosis of heart murmurs in the heart sound signal with the graphical display of sub-interval mid-range energy magnitude and murmur contour helps provide the physicians with the tools to make a diagnosis of disease pathology or further refer the patient for more detailed testing. For example, AHA guidelines for echocardiography referral includes having the physician 1) determine if a murmur is present, 2) whether it is in systole or diastole. If it is in systole, whether it is soft or loud and its contour. With auscultation alone, this is done entirely by listening. The present invention provides a graphical means for assertion of murmur presence, location, magnitude and contour.

### Exemplary Embodiments

1. Hypertrophic Cardiomyopathy Diagnosis

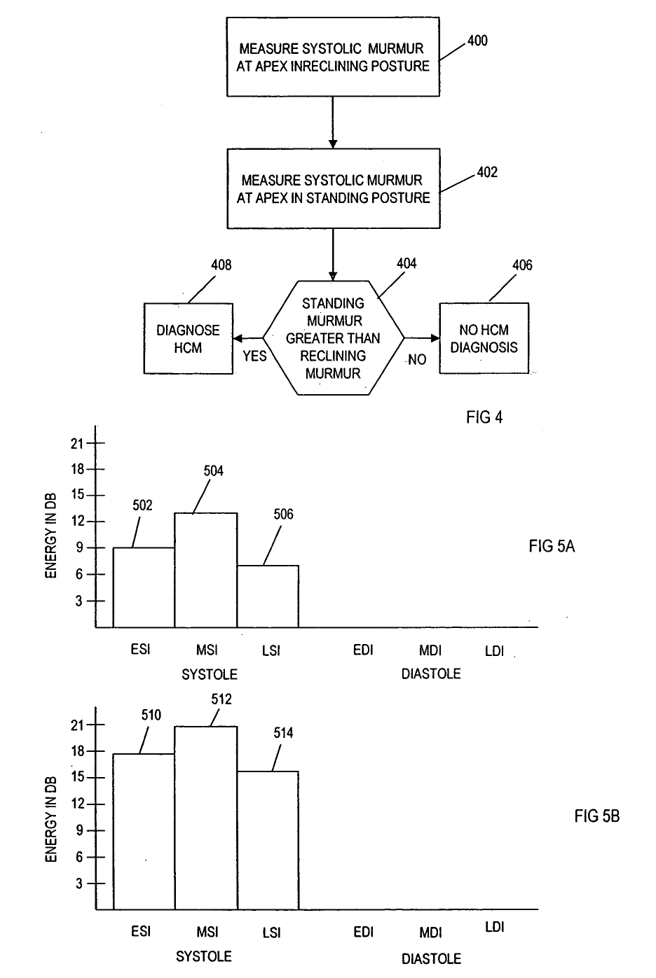

FIG. 4 represents an application of the method of the present invention to diagnosing hypertrophic cardiomyopathy (HCM), both nonobstructive and obstructive. A physician desirably applies an electronic stethoscope to a patient's apex, a standard auscultation site, and measures the murmur in the reclining position, step 400. The physician may next have the patient switch postures to a standing position and again measures the murmur at the apex position, step 402.

To diagnose HCM, the systolic murmur intensity in the standing posture is compared to the intensity in the reclining posture, step 404. If the systolic murmur intensity on standing is greater than the intensity on reclining then an affirmative HCM diagnosis, step 408 may be made. If the systolic murmur intensity on standing is not greater than the reclining intensity, there may be no conclusive diagnosis of HCM, step 406.

The present invention may be used to determine and compare the presence of heart murmurs from each posture. The heart sound signal received from an electronic stethoscope is processed for both postures. Heart murmurs may be diagnosed by incorporating a mid-range energy measure into a murmur detection algorithm. In addition, the mid-range energy may be displayed as a bar graph showing the sub-systolic and sub-diastolic energy and contour.

For example, the resulting energy magnitude shown in FIGS. 5A and 5B may be indicative of diagnosing HCM. If FIG. 5A represents the energy received while a patient is in the reclining position and FIG. 5B represents the energy received during standing, the patient may be diagnosed as having HCM.

2. Adjusting Therapeutic Drug

Systolic and/or diastolic murmurs may occur with various heart conditions, including those conditions that may be treated with medication. Heart murmurs may typically be discovered during auscultation in a physical exam. A physician may assign the murmur a subjective grade for murmur magnitude. The grading and typifying, e.g. early grade 3 systolic, are based upon listening to the heart and may typically vary by physician and by exam. There is no objective record to review heart murmur details. Lack of an objective heart murmur measure may cause difficulty in adjusting therapeutic drug dosage to reduce heart murmurs.

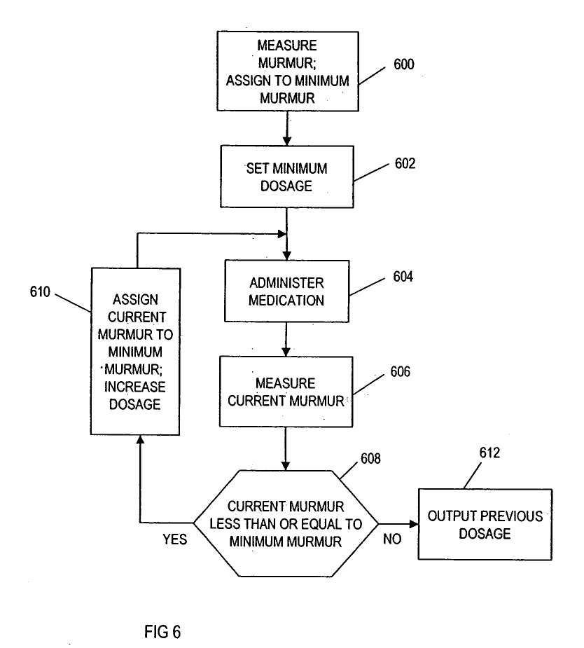

In FIG. 6, the present invention may be used to adjust therapeutic drug dosage. In step 600, a heart sound recording is initially made to determine the presence and magnitude of a heart murmur. The heart signal is received, processed and parsed for heart murmurs using a murmur detection algorithm in conjunction with a mid-range energy measure. Mid-range energy is desirably displayed as a bar graph. This initial murmur diagnosis and energy is assigned to a minimum murmur value.

Based upon the initial murmur magnitude and other factors such as disease, age, weight and so forth, a minimum dosage may be determined, step 602. After the minimum dosage is administered, step 604, a stabilization period may be required for the medication to take effect.

After a stabilization period, the heart murmur is again measured, step 606. Because an objective record has been kept of the initial measurement, a different healthcare professional may make the new recording without subjectively skewing the resultant analysis. The recorded murmur diagnosis and magnitude of the set minimum murmur is compared against this new murmur diagnosis and magnitude, step 608.

If the current murmur is less than or equal to the initial minimum murmur of step 600, this new murmur is assigned as the minimum murmur and the medication dosage may be adjusted, step 610. The medication is administered again, step 604. The murmur is measured again after any required stabilization period, step 606, and the new murmur state and the minimum murmur state are compared, step 608.

If the current murmur is not less than or equal to a minimum murmur, the previous medication dosage is kept, step 612 and the heart condition may be controlled. The dosage may be monitored and increased using steps 604, 606, 608 and 610 until a desired murmur decision and magnitude is achieved.

3. Adjusting Therapeutic Device

It may be desirable to provide an objective measure for adjusting a therapeutic device, such as a pacemaker. It is often difficult to adjust the device. Specifically, it may be difficult for a physician to make a judgment as to whether murmur loudness is decreased.

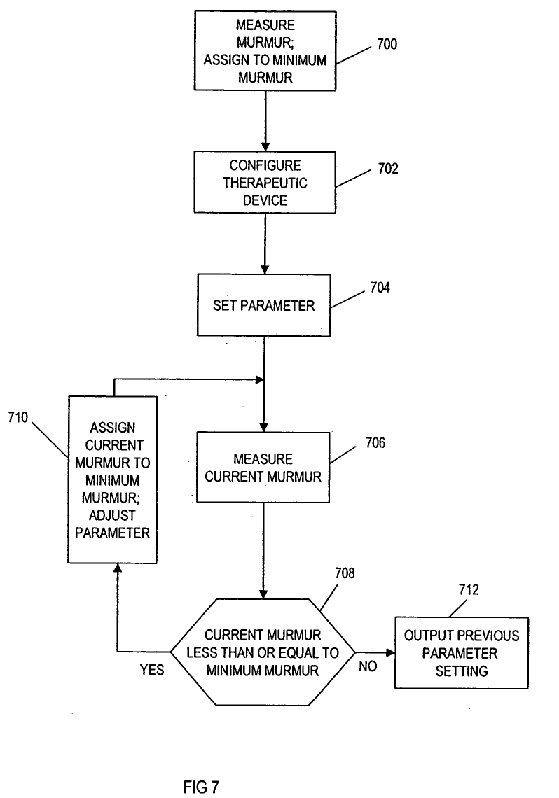

In FIG. 7, the present invention is applied to determining a therapeutic device parameter setting. In step 700, a heart sound recording is initially made to determine the presence and magnitude of a heart murmur. The heart signal is received, processed and parsed for heart murmurs. A murmur detection algorithm is conjoined with a mid-range energy measure to diagnose the heart signal for heart murmurs. A mid-range energy is also desirably displayed as a bar graph. This initial murmur decision is assigned to a minimum murmur value. Based upon the initial murmur decision and other physiological factors, the therapeutic device may be initially configured, step 702.

After a parameter is set, step 704, the heart murmur may again be measured, step 706. The recorded murmur diagnosis and energy of the minimum murmur is compared against this new murmur diagnosis and magnitude, step 708.

If the current murmur is less than or equal to the minimum murmur, then this new murmur is assigned as the minimum murmur and the therapeutic device parameter may be adjusted, step 710. The murmur is measured again, step 706, and a comparison of the new murmur state and the minimum murmur, step 708.

If the current murmur is not less than or equal to a minimum murmur, the previous therapeutic device parameter value is kept, step 712 and the heart condition may be controlled. The parameter value may be monitored and adjusted using steps 706, 708 and 710 until a desired murmur state is achieved.

Although the invention has been described as a method, it is contemplated that it may be practiced by a general purpose computer configured to perform the method or by computer program instructions embodied in a computer-readable carrier such as an integrated circuit, a memory card, a magnetic or optical disk or an audio-frequency, radio-frequency or optical carrier wave.

Although the invention is illustrated and described herein with reference to specific embodiments, the invention is not intended to be limited to the details shown. Rather, various modifications may be made in the details within the scope and range of equivalents of the claims and without departing from the invention.

What is claimed:

1. A method for assisting in the diagnosis of heart murmurs, the method comprising the steps of:
calculating a normalized measure of mid-range energy for at least one of systolic or diastolic intervals in a sequence of heartbeats signals wherein the mid-range energy is between 150 and 600 Hz; and
displaying the mid-range energy measure of the at least one systolic or diastolic interval in a graphical form.

2. The method of claim 1 wherein the step of displaying the mid-range energy measure of the at least one systolic or diastolic interval includes the step of displaying the mid-range energy level as a bar graph.

3. The method of claim 2 further comprising the step of converting the mid-range energy into a murmur grade and displaying an indication of the murmur grade.

4. The method of claim 1 wherein the mid-range energy is between 206 Hz and 566 Hz.

5. The method of claim 1 wherein the step of calculating a normalized, mid-range energy further comprises the step of processing the audio signals using a Morlet wavelet.

6. The method of claim 1 wherein:
the step of calculating the normalized measure of mid-range energy includes the steps of:
dividing the at least one of the systolic or diastolic intervals into a plurality of subintervals;
calculating respective measures of the mid-range energy for each of the subintervals; and
the step of displaying the mid-range energy comprises the step of graphically displaying the calculated mid-range energy measures for each of the plurality of sub-intervals.

7. The method of claim 6 wherein:
the step of dividing the at least one of the systolic or diastolic intervals into a plurality of subintervals includes the steps of:
subdividing the systolic intervals into at least three time intervals;
subdividing the diastolic intervals into at least three time intervals.

8. A method for assisting in the diagnosis of heart murmurs, the method comprising the steps of:
receiving an audio signal representing heart sounds;
detecting heart murmurs in the audio signal during a predetermined interval to develop a count of the murmurs;
processing the audio signal to develop a measure of mid-range energy wherein the mid-range energy is in a range between 150 and 600 Hz;
adjusting a murmur count threshold responsive to the measure of mid-range energy; and
diagnosing a heart condition responsive to the murmur count and the adjusted murmur count threshold.

9. The method of claim 8 wherein the mid-range energy is in a range between 206 Hz and 566 Hz.

10. The method of claim 8 further comprising the step of adjusting the murmur count threshold in an inverse proportion to the measure of mid-range energy.

11. The method of claim 10 further comprising the step of translating the mid-range energy into a murmur grade and adjusting the murmur count threshold responsive to the murmur grade.

12. The method of claim 10 wherein the step of diagnosing the heart condition includes the step of diagnosing a murmur in one of the systolic and diastolic intervals.

13. A method for determining an effective dose of a therapeutic agent for treating a heart condition, the method comprising the steps of:
(a) administering a trial dosage of the therapeutic agent;
(b) receiving an audio signal representing heart sounds;
(c) detecting heart murmurs in the audio signal during a predetermined interval to develop a count of the murmurs;
(c) processing the audio signal to develop a measure of mid-range energy wherein the mid-range energy is in a range between 150 and 600 Hz;
(d) adjusting a murmur count threshold responsive to the measure of mid-range energy; and
(e) determining an effectiveness of the trial dose of the therapeutic agent responsive to the murmur count and the adjusted murmur count threshold;
(f) repeating steps (a) through (e) until the effective dose of the therapeutic agent is determined.

14. A method for adjusting a parameter of a therapeutic device to treat a heart condition, the method comprising the steps of:
(a) setting the parameter to a trial setting;
(b) receiving an audio signal representing heart sounds;
(c) detecting heart murmurs in the audio signal during a predetermined interval to develop a count of the murmurs;
(c) processing the audio signal to develop a measure of mid-range energy wherein the mid-range energy is in a range between 150 and 600 Hz;
(d) adjusting a murmur count threshold responsive to the measure of mid-range energy; and
(e) determining an effectiveness of the trial parameter setting responsive to the murmur count and the adjusted murmur count threshold.
(f) repeating steps (a) through (e) until an effective value of the parameter is determined.

15. The method of claim 14 wherein the therapeutic device is a pacemaker.

16. A method for diagnosing cardiac murmurs associated with hypertrophic cardiomyopathy (HCM) in a patient, the method comprising the steps of:
determining presence and magnitude of cardiac murmurs in the patient while the patient is in a plurality of posture;
comparing the determined magnitude of cardiac murmurs in the plurality of postures to diagnose HCM in the patient;
wherein the step of determining the presence and magnitude of cardiac murmurs comprises the steps of:
receiving an audio signal representing heart sounds;
detecting cardiac murmurs in the audio signal during a predetermined interval to develop a count of the murmurs;
processing the audio signal to develop a measure of mid-range energy wherein the mid-range energy is in a range between 150 and 600 Hz, the measure of mid-range energy represents the magnitude of the cardiac murmurs;
adjusting a murmur count threshold responsive to the measure of mid-range energy; and
determining the presence of cardiac murmurs responsive to the murmur count and the adjusted murmur count threshold.

17. The method of claim 16 wherein the mid-range energy is between 206 Hz and 566 Hz.

18. The method of claim 16 wherein the step of determining presence of cardiac murmurs in the first and second postures includes generating the audio signal representing the heart sounds by applying an electronic stethoscope to the apex measurement point of the patient.

19. The method of claim 18 wherein the plurality of postures includes an upright posture and a reclining posture.

20. A method for detecting cardiac murmurs, the method comprising the steps of:
receiving an audio signal representing heart sounds;
processing the audio signal using a hidden Markov model HMM to identify heart beats and cardiac murmurs within the heart beats;
calculating respective counts of heart beats and heart beats with murmurs detected during the predetermined interval;
computing a duration for each detected heart beat and a median duration for all heart beats detected during a predetermined interval;
comparing the count of heart beats detected during the predetermined interval to a number of heart beats that should have been detected based on the median duration and the predetermined interval to calculate a beat detection ratio;
calculating a murmur count threshold responsive to the calculated beat detection ratio;
processing the audio signal to develop a measure of mid-range energy wherein the mid-range energy is in a range between 150 and 600 Hz;
adjusting the murmur count threshold responsive to the measure of mid-range energy; and
determining the presence of cardiac murmurs responsive to the murmur count and the adjusted murmur count threshold.

21. The method of claim 20 wherein the step of adjusting the murmur count threshold adjusts the threshold in inverse proportion to the measure of mid-range energy.

22. The method of claim 20 and further including processing the audio signal using a neural network to identify heart beats and cardiac murmurs within the heart beats.

23. The method of claim 22, wherein the neural network is of time-delay type.

24. The method of claim 20 and further including processing the audio signal using MEL cepstrum signal analysis to identify heart beats and cardiac murmurs within the heart beats.

25. A computer readable medium including computer program instructions adapted to instruct a general purpose computer to perform a method for assisting in the diagnosis of heart murmurs in response to a received audio signal representing heart sounds, the method comprising the steps of:
detecting heart murmurs in the audio signal during a predetermined interval to develop a count of the murmurs;
processing the audio signal to develop a measure of mid-range energy wherein the mid-range energy is in a range between 150 and 600 Hz;
adjusting a murmur count threshold responsive to the measure of mid-range energy; and
diagnosing a heart condition responsive to the murmur count and the adjusted murmur count threshold.

26. The computer readable medium of claim 25 wherein the computer program instructions which instruct the general purpose computer to perform step of processing the audio signal instruct the computer to process the mid-range energy in a range between 206 Hz and 566 Hz.

27. The computer readable medium of claim 25 wherein the computer program instructions which instruct the general purpose computer to perform step of adjusting the murmur count threshold instruct the computer to adjust the murmur count threshold in an inverse proportion to the measure of mid-range energy.

28. The computer readable medium of claim 25 further comprising computer program instructions adapted to instruct the general purpose computer to perform the step of translating the mid-range energy into a murmur grade, wherein the computer program instructions that are adapted to instruct the general purpose computer to adjust the murmur count threshold, cause the computer to adjust the murmur count threshold responsive to the murmur grade.
